(19) United States
(12) Patent Application Publication
(10) Pub. No.: US 2024/0139101 A1
(43) Pub. Date: May 2, 2024

(54) ADVANCED ORAL FILM FORMULATIONS

(71) Applicant: INTELGENX CORP., ST-LAURENT (CA)

(72) Inventors: Rima CHOUIKRAT, St-Laurent (CA); Nadine PAIEMENT, St-Laurent (CA)

(73) Assignee: INTELGENX CORP., ST-LAURENT, QC (CA)

(21) Appl. No.: 18/386,113

(22) Filed: Nov. 1, 2023

Related U.S. Application Data
(60) Provisional application No. 63/421,460, filed on Nov. 1, 2022.

(51) Int. Cl. A61K 9/00; A61K 31/00; A61K 36/185; A61K 47/10; A61K 47/14; A61K 47/26; A61K 47/32; A61K 47/34; A61K 47/38

(52) U.S. Cl. CPC A61K 9/006; A61K 31/658; A61K 36/185; A61K 47/10; A61K 47/14; A61K 47/26; A61K 47/32; A61K 47/34; A61K 47/38

(57) ABSTRACT

This disclosure relates to oral film dosage formulations and processes for preparing oral film dosage forms for the delivery of poorly water-soluble drugs and more particularly to the preparation of oral film dosage forms that are suitable for cannabis drug delivery.

### CROSS-REFERENCE TO RELATED APPLICATIONS

The present application claims priority to U.S. Application No. 63/421,460 filed Nov. 1, 2022. This document is hereby incorporated by reference in its entirety.

### FIELD OF THE DISCLOSURE

This disclosure relates to oral film dosage formulations and processes for preparing oral film dosage forms for the delivery of poorly water-soluble drugs and more particularly to the preparation of oral film dosage forms that are suitable for cannabis drug delivery.

### BACKGROUND OF THE DISCLOSURE

Oral delivery is a non-invasive and convenient route of administration. Many people have difficulty swallowing tablets and capsules, and risk choking while attempting to swallow solid oral dosage forms, but can self-administer a film dosage form without difficulty. Oral films are promising for use in the pediatric and geriatric populations, as well as in veterinary administration.

Cannabinoids are natural extracts from the plant Cannabis sativa. Research has focused more on delta-9-tetrahydrocannabinol (THC) and cannabidiol (CBD). THC is known to cause psychoactive effects or the ‘high’ felt from cannabis. Actually, it is the metabolite of delta 9 THC the 11-hydroxy THC that is responsible for the psychoactive effect. This is also why a bucally absorbed THC, which would limit metabolization and reduce 11-OH-THC would be beneficial.

Sativex is a buccal spray 1:1 THC and CBD approved and on the market. Sativex is indicated as treatment for symptom improvement in adult patients with moderate to severe spasticity due to multiple sclerosis (MS) who have not responded adequately to other anti-spasticity medication and who demonstrate clinically significant improvement in spasticity-related symptoms. THC has proven beneficial in patients suffering from Post-Traumatic Stress Disorder [PTSD], as an appetite stimulant for patients with HIV/AIDS, in reducing nausea and vomiting in patients on chemotherapy. On the other hand, CBD lacks nearly any psychoactive effect and has shown promise in treating epilepsy, including a severe form of epilepsy in children called Dravet's syndrome. CBD has also been used successfully by patients with genetic brain disorders, Crohn's disease and ulcerative colitis, and Parkinson's disease.

Conventional methods of preparing and ingesting cannabis suffer from drawbacks. For example, smoking cannabis necessarily creates harmful carcinogens through pyrolysis of numerous plant compounds, as well as irritation to the lung tissue. Similarly, eating cannabis requires a significant period of time before the onset of effects, and the uptake through the gastrointestinal tract is uneven and incomplete.

THC and CBD undergo significant hepatic-first-pass metabolism resulting in bioavailability about 2-8% hence delivery through the sublingual and buccal pathways is preferred to improve dosing and bioavailability, especially for patients requiring quick relief.

THC and CBD are lipophilic compounds which makes their incorporation into hydrophilic polymers challenging. Additionally, when extracted from cannabis as distillate they have a distinct bitter taste which makes them unpalatable to many patients, the taste is derived from the terpenes, flavonoids and other molecules extracted and present in the THC and CBD distillate. In addition, the distillate is a semi-solid resin that requires heat to be become liquid increasing the manufacturability difficulties.

There is a need for pharmaceutically acceptable oral disintegrating films that address the issues associated with effective absorption of the cannabis-related active. There is also a need to make a THC film where the API is buccally absorbed, leading to a faster onset of action and a lower metabolization of the API into the main metabolite 11-hydroxy THC to reduce adverse events associated with metabolite-like hallucination.

These and other inefficiencies and opportunities for improvement are addressed and/or at least partially overcome by the systems, assemblies and methods of the present disclosure.

### BRIEF DESCRIPTION OF THE DRAWINGS

FIG. 1 illustrates the difference in dissolution profiles between example 1 and example 3, using 10 mg THC films.

FIG. 2 illustrates the difference in dissolution profiles between example 2 and example 3, using 10 mg THC films.

FIG. 3 illustrates the difference in dissolution profiles between example 4 and example 5, using 10 mg THC films.

FIG. 4 illustrates the difference in dissolution profiles between example 6 and example 7, using 10 mg THC films.

FIG. 5 illustrates the difference in dissolution profiles between example 8 and example 9, using 10 mg THC films.

FIG. 6 illustrates the difference in dissolution profiles between example 10 and example 11, using 10 mg THC films.

FIG. 7 illustrates the difference in dissolution profiles between example 1, example 2, example 5, example 7, example 9 and example 11, using 10 mg THC films.

FIG. 8 illustrates the difference in dissolution profiles between example 1, example 5, example 7 and example 13, using 10 mg THC films.

### SUMMARY OF THE DISCLOSURE

In general, an oral dosage form for buccal absorption includes amorphous cannabis, a solvent system, a permeation enhancer, solubilizer, a mucoadhesive polymer and a sweetener.

In one of its aspects, this disclosure relates to stable cannabinoids sublingual or buccal film with fast disintegration, high dissolution and mucoadhesive properties. This new strategy formulation promotes systemic exposure by supporting the release of the cannabinoids sublingually and buccally, accelerating the onset of action and enhancing the oral bioavailability.

According to some aspects of the disclosure, the oral dosage form disclosed herein involves an equilibrium between solubilizing the cannabis using solvent(s) or an emulsion embedded in a matrix formulation that stabilizes but does not entrap the cannabis.

According to some aspects of the disclosure, the cannabis is primarily amorphous, which is defined as 75% or more of the cannabis in its amorphous state.

According to some aspects of the disclosure, the mucoadhesive polymer is sodium carboxymethyl cellulose or polycarbophil.

According to some aspects of the disclosure, the primary mucoadhesive polymer is HPMC or Sodium Alginate.

According to some aspects of the disclosure, the secondary mucoadhesive polymer is HPMC, HPC, HPC/copovidone combination or HPC/HPMC combination.

According to some aspects of the disclosure, the sweetener is sucralose, sorbitol or maltitol.

According to some aspects of the disclosure, the oral dosage form further comprises a permeation enhancer.

According to some aspects of the disclosure, the oral dosage form further comprises a solubilizer.

According to some aspects of the disclosure, the oral dosage form further comprises an emulsifier.

According to some aspects of the disclosure, the oral dosage form further comprises a surfactant.

According to some aspects of the disclosure, the oral dosage form further comprises a stabilizer.

According to some aspects of the disclosure, the oral dosage form further comprises a plasticizer.

According to some aspects of the disclosure, the oral dosage form further comprises a flavor.

In certain aspects of this disclosure, the disclosed formulations and excipients are specifically adapted for use in humans.

In certain aspects of this disclosure, the disclosed formulations and excipients are specifically adapted for use in animals.

In certain aspects of this disclosure, the disclosed excipients are food grade. And the process manufacturing process follows good production practices (GPP).

These and other features, advantages and objects of the various embodiments will be better understood with reference to the following specification and claims.

### DETAILED DESCRIPTION

Unless defined otherwise, all technical and scientific terms used herein have the same meaning as commonly understood by one of ordinary skill in the art.

References in the specification to “one embodiment,” “an embodiment,” “an example embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to effect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “therapeutically effective amount” refers to an amount of a pharmaceutically active agent, which when administered to a particular subject, considering the subject's age, weight and other relevant characteristics, will attenuate, ameliorate, or eliminate one or more symptoms of a disease or condition that is treatable with the pharmaceutically active agent.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additionally, unrecited elements or method steps.

Film systems embody a field of technology that has major advantages in areas of administering various actives to an individual or subject in need thereof. The present disclosure relates to oral films, also referred to in the art as films and film strips, sheets, discs, wafers, and the like, in any shape, including rectangular, square, or other desired shape, and methods for forming film products that include at least one active. Specifically, the disclosure provides for a film and a method of forming a film. The terms “oral dissolving film,” “oral dissolvable film”, “oral disintegrating film”, OSF, “oral soluble film”, “ODF”, “oral chewable film”, “OCF”, “oral dissolvable film”, “OTF,” “oral drug strip” or “oral strip” refer to a product used to administer a predetermined amount of active ingredient(s) via oral administration such as oral transmucosal absorption, sublingual delivery or buccal delivery and will be referred to throughout as “oral film(s)”.

The term “active agent(s)” or “API” refers mainly to active pharmaceutical ingredients, drugs, pharmaceuticals, but may also refer generally to any agent(s) that chemically interacts with the subject to which it is administered to cause a biological change, such as, but not limited to, eliminating symptoms of disease, condition or regulating biological functions.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug.

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

The film product of the present disclosure includes an active component selected from pharmaceutical agents, medicaments, drugs, bioactive agents, cosmetic agents, food grade and combinations thereof. The active component may be present in any desired amount effective for the intended treatment. It is particularly desirable and an advantage of the present disclosure that the active component can be included at high loading.

The term “therapeutically effective amount” refers to an amount of a pharmaceutically active agent, which when administered to a particular subject, considering the subject's age, weight and other relevant characteristics, will attenuate, ameliorate, or eliminate one or more symptoms of a disease or condition that is treatable with the pharmaceutically active agent.

A variety of additives that can be integrated into the films may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added along with the active ingredient(s).

As used herein, “Cannabis” refers to a genus of flowering plants that includes a single species, Cannabis sativa, which is sometimes divided into two additional species, Cannabis indica and Cannabis ruderalis. Cannabis has long been used for fiber (hemp), for seed and seed oils, for medicinal purposes, and as a recreational drug. Various extracts including hashish and hash oil are also produced from the plant. The Cannabis can include any physical part of the plant material, including, e.g., the leaf, bud, flower, trichome, seed, or combination thereof. Likewise, Cannabis can include any substance physically derived from Cannabis plant material, such as hashish.

As used herein, “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid 09-tetrahydrocannabinol (THC), the primary psychoactive compound of Cannabis. Cannabidiol (CBD) is another major constituent of the plant, representing up to 40% in extracts of the plant resin. There are at least 85 different cannabinoids isolated from Cannabis, exhibiting varied effects. Pharmacologically active cannabinoids are classified as Class II according to the Biopharmaceutics classifications system (BCS), i.e. poorly water-soluble and highly permeable due to their crystalline hydrophobic characteristics which results in poor bioavailability after oral administration. The rate at which the cannabis enters the bloodstream through the oral mucosa is an important characteristic and may have an influence on the biological response elicited by the cannabis; class II substances are limited by the dissolution process rather than the absorption process. Slow dissolution of cannabis results in incomplete, erratic and unpredictable absorption. Manipulating the formulation (ie, drug form, adding solubilizers, solvents) can change the dissolution rate and thus control overall absorption. Cannabinol (CBN): CBN is a mildly psychoactive cannabinoid that forms when THC oxidizes. It may have sedative properties and is often associated with aged cannabis. This disclosure considers all different cannabinoids including the following: Tetrahydrocannabinolic Acid (THCA): THCA is the precursor to THC and is not psychoactive until it is decarboxylated (usually by heat). It may have anti-inflammatory properties. Cannabidiolic Acid (CBDA): CBDA is the acidic precursor to CBD and has potential anti-nausea and anti-inflammatory effects. Cannabigerolic Acid (CBGA): CBGA is the precursor to both THC and CBD and plays a role in the biosynthesis of other cannabinoids. Cannabidivarin (CBDV): CBDV is structurally similar to CBD and may have anti-epileptic and anti-nausea properties.

Tetrahydrocannabivarin (THCV): THCV is a psychoactive cannabinoid with effects that may vary depending on the dose. It's been explored for its potential appetite-suppressing properties. Cannabichromevarin (CBCV): Similar to CBC, CBCV is a non-psychoactive cannabinoid that may have anti-epileptic properties. Cannabigerolvarin (CBGV): CBGV is a lesser-known cannabinoid with potential anti-inflammatory properties. Cannabicyclol (CBL): CBL is a non-psychoactive cannabinoid with limited research, but it may have some potential therapeutic effects.

The term “matrix” or “film matrix” refers to the polymer component or mixture of polymers, which creates the “film-forming matrix” supporting the API within the oral film dosage form.

The term “amorphous” refers to the non-crystalline form of the solid, a state that lacks the regular crystalline organization of atoms. The amorphous content (amorphicity) of a solid can be accurately and precisely assessed using a number of well-established methodologies, including isothermal calorimetry, Powder X-ray Diffraction (PXRD), Differential Scanning calorimetry (DSC), Continuous Relative Humidity Perfusion, Microcalorimetry (cRHp), and Dynamic Vapor Sorption (DVS).

The term “mucoadhesive” and variations thereof generally refers to film matrix or pharmaceutical dosage form interacting by means of adhesion with the mucus that covers epithelia.

The term “mucoadhesive film former” refers to polymers that form the film matrix, film strip, film sheet and dissolves in aqueous environment and gives bio-adhesive properties to the mucosa examples comprising PEO, Pullulan, CMC, HPC, HPMC, chitosan, sodium alginate and ethyl cellulose (EC), polyvinyl alcohol (PVA), Starch, Polymethacrylate polymers. Examples of mucoadhesive materials that can be used to prepare the mucoadhesive film matrix include poly(ethylene oxide), polyvinyl pyrrolidone, poly(acrylic acid) derivatives (e.g., commercially available Carbopol®), polycarbophil, polyoxyalkylene ethers, polymethacrylates, polymethacrylates-based copolymers (e.g., commercially available Eudragit®), biodegradable polymers such as poly(D,L-lactide-co-glycolide) (e.g., commercially available Resomer®), anionic biopolymers such as hyaluronic acid, or sodium carboxymethylcellulose, cationic biopolymers such as chitosan or poly(L-lysine) and other cellulose derivatives. Other mucoadhesive polymers that can be used include methyl vinyl ether-maleic acid, a mixed salt of sodium/calcium methyl vinyl ether-maleic acid, methyl vinyl ether-maleic anhydride, gums including xantan gum, arabix gum and guar gum, polyglycolic acid (PGA) and half esters (monoethyl; monobutyl and isopropyl ester) of methyl vinyl ether-maleic anhydride copolymers (e.g., commercially available Gantrez®).

The term “bioavailability” will have its meaning as prescribed in the art, as the ability of a drug or other substance to be absorbed and used by the body. Bioavailability is an important factor in oral film technology. The sublingual mucosa has high membrane permeability due to its thin membrane structure and high vascularization. Due to this rapid blood supply, it offers very good bioavailability. Enhanced systemic bioavailability limits first-pass effect and better permeability is owing to high blood flow and lymphatic circulation. In addition, the oral mucosa is a very effective and selective route of systemic drug delivery because of the large surface area and ease of application for absorption. In studies, thin films have shown their abilities such as improving the initial effect of the drug and duration of this effect, decreasing the frequency of dosing, and increasing the effectiveness of the drug.

The term “emulsifier or “emulsifying agent” is a chemical compound that permits the mixing of two or more immiscible liquids. In addition to promoting the blending of dissimilar compounds, emulsifying agents are also responsible for keeping the mixture stable, i.e., preventing the individual elements from separating.

The term “permeation enhancer” and variations thereof generally refers to is a chemical compound which is added into the formulation along with the target drug in order to improve permeation through the biological membrane such as the skin, nasal, and intestinal mucosae. Examples comprising but not limited to bile salts, fatty acids and derivatives, glycerides, chitosan, surfactants, cyclodextrins derivatives, pH modulators, and mucoadhesive excipients.

The term “flavoring Agent” or flavor” and variations thereof generally refers to concentrated preparations, with or without flavor adjuncts required in their manufacture, used to impart flavor, with the exception of salt, sweet, or acid tastes. Flavoring agents may be classified as natural, artificial, or natural and artificial (N&A) by combining the all-natural and synthetic flavors or other forms known in the art. Flavoring agents are categorized by their physical classification as solid flavoring agents and liquid flavoring.

The term “flavor enhancer” and variations thereof generally refers to compounds that particularly enhance certain tastes or reduce undesirable flavors without having an especially strong taste of their own. Flavor enhancers harmonize taste components and make food/drug preparations more palatable. Examples include but are not limited to maltol, ethyl maltol and monosodium glutamate, glutamic acid, glutamates, purine-5-ribonucleotides, inosine, guanosine, adenosine 5_-monophosphates, sugars, sweetener, carboxylic acids (e.g., citric, malic, and tartaric), common salt (NaCl), amino acids, some amino acid derivatives (e.g., monosodium glutamate—MSG), and spices (e.g., peppers) are most often employed, yeast, yeast extract, dried yeast and others or mixtures thereof.

The term “sweetener” or “sweetening agent” and variations thereof generally refers to a solid or liquid ingredient that is used to impart a sweet taste to food or drug product. Sweeteners are often classified as either nutritive (caloric) or non-nutritive (non-caloric), natural or synthetic. Examples of sweeteners include but are not limited to sucrose, dextrose, lactose, glucose, advantame, sorbitol, mannitol, liquid glucose, honey molasses, saccharin, sucralose, rebaudioside A stevia, rebaudioside M stevia, stevioside, mogroside IV, mogroside V, alitame, saccharin, neohesperidin dihydrochalcone, cyclamate, neotame, N-[3_(3-hydroxy-4-methoxybenzyl yl) propyl]-L-α-aspartyl]-L-phenylalanine 1-methyl ester, N-[3-(3-hydroxy-4-methoxyphenyl)-3-methylbutan yl]-L-α-aspartyl]-L-phenylalanine 1-methyl ester, N-[3-(3-methoxy-4-hydroxyphenyl) propyl]-L-α-aspartyl]-L-phenylalanine 1-methyl ester, curculin, cyclamate, aspartame, acesulfame potassium and others or mixtures thereof.

The term “carrier oil” or “base oil” generally refers to a liquid ingredient that is used to dilute and solubilise fat molecules and improve their bioavailability. Fat-soluble substances are better absorbed when digested along with fat, even in small amounts. Carrier oils are also needed to ensure the lipophilic drug remains stable and potent for longer. Carrier oils oxidize at a slower rate, they retain the freshness of the compounds within and increase the lipophilic drug's shelf life. Examples of carrier oils include but are not limited to Medium Chain triglyceride MCT oil, Caprylic/Capric Triglyceride Glycerol (Captex 355), olive oil, sesame oil, avocado oil, coconut oil, oleic acid, linoleic acid, castor oil, low HLB emulsifiers can act as carrier oils in emulsions like Capmul MCM C8.

The term “surfactant” refers to excipients that are employed to dissipate the free surface energy of particles by reducing the interfacial tension and contact angle between the solid and the suspending vehicle and comprise PEG 300 oleic glycerides (Labrafil® M-1944CS), PEG 300 linoleic glycerides (Labrafil® m-2125CS); Hydroxylated lecithin; Caprylocaproyl polyoxyl-8 glycerides; Polyoxyethylene (4) sorbitan monostearate, Polyoxyethylene 20 sorbitan tristearate, Polyoxyethylene (5) sorbitan monooleate, Polyoxyethylene 20 sorbitan trioleate; Sorbitan Esters (Sorbitan Fatty Acid Esters) such as: Sorbitan monolaurate, Polyoxyethylene Sorbitan Fatty Acid Esters such as Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene 20 sorbitan monooleate, Polyoxyethylene 20 sorbitan monoisostearate Polyethylene glycol monostearate (Gelucire 48/16), poloxamer having MW up to 14.600, viscosity up to 3100 mPAs (77 C) but exclude surfactant(s) of an HLB below 7 such as Propylene glycol monocaprylate type I, Propylene glycol monocaprylate type II, Propylene glycol monolaurate, Sorbitan monoisostearate, Sorbitan monooleate, Sorbitan monopalmitate, Sorbitan monostearate, Sorbitan sesquioleate, Sorbitan trioleate, Brij®, Polyoxyethylene lauryl ether, Polyethyleneglycol lauryl ether Sorbitan tristearate, glyceryl monoleate.

The term “co-surfactant” refers to a chemical substance that is used in addition to a surfactant to improve its performance especially: a second surfactant that is used in conjunction with a primary surfactant. Co-surfactants are usually alcohols or amines ranging from C4 to C10 and helps in the formation and stabilization of micelles/microemulsions.

The term “plasticizer” as used herein refers to a substance that produces or promotes plasticity and flexibility and to reduce brittleness. Plasticizers can be advantageously employed in film formulations as needed to suitably modify the flexibility of the film to facilitate processing and allow the film to easily conform to the shape of the oral mucosa to which the film is applied. Plasticizers may reduce the glass-transition temperature of the film forming polymers (e.g., the water-soluble polymer or water-soluble polymers in the film). Examples of plasticizers that can be used in the disclosed oral film dosage forms include triacetin, triethyl citrate, tributyl citrate, acetyl tributyl citrate, acetyl triethyl citrate, trioctyl citrate, acetyl trioctyl citrate, trihexyl citrate, dibutyl sebacate, PEG 300, PEG 400, Glycerine, Propylene glycol etc. Plasticizer may be added in an amount up to 25%, alone or as a combination, of the total mass of the film oral dosage form, such as from 0.5% to 25%, 1% to 20%, 2% to 15% or 5% to 10%.

The term “stabilizer” refers to a substance which prevents degradation of the product. An example of stabilizer is an “antioxidant”, which prevents or inhibits oxidation of molecules by terminating free radical reactions, and may delay or prevent some types of cellular damage. Antioxidants may be naturally occurring including those found in foods and botanical materials or synthetic. Non-limiting examples of antioxidants include citric acid, Vitamin E, vit E-D-α-tocopheryl polyethylene glycol succinate or a derivative thereof, a tocopherol, and combinations thereof. In some embodiments, the antioxidant is Vitamin E or a derivative thereof, a flavonoid, a polyphenol, a carotenoid, or a combination thereof. Other stabilizer examples includes but are not limited to butylated hydroxyanisole (BHA), butylated hydroxytoluene (BHT), tertiary butylhydroquinone (TBHQ), phenolic acids, Ethylenediaminetetraacetic acid (EDTA), sodium metabisulfite, cupper, guthathion, vitamin C and derivation like ascorbic palmitate or a combination thereof.

The term “preservative” refers to an agent that extends the storage life of food and non-food products by retarding or preventing deterioration of flavor, odor, color, texture, appearance, nutritive value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involves mode of action (a preservative prevents growth rather than killing microorganisms) and exposure time (a preservative has days to months to act whereas a sanitizer has at most a few minutes to act). In specific embodiments, the preservative includes at least one of sodium benzoate, methyl paraben, propyl paraben, and sodium sorbate.

The term “surface pH” refers to the pH measured on a surface of the film, such as the top or bottom surface of a monolayer film or on an exposed surface of the layer containing the active in a multilayer oral film. The film is prepared for pH testing by slightly wetting the film (adding water as needed for a pH test—e.g. one to three drops). The pH is then measured by bringing the surface electrode in contact with the surface of the oral film. This measurement of the surface pH is preferably performed on several films of the same formulation.

The terms “blend” or “blending media” and variations thereof generally refers to the combination of the OF formulation with the presence of solvents.

The term “drug absorption” or “absorption” as used in this specification, refers to the process of movement from the site of the administration of a drug toward the systemic circulation, e.g., into the bloodstream of a subject.

The term “residence time” as used in the specification refers to the time taken by the film to disintegrate on the sublingual or buccal mucosa.

The term “instantly wettable” and variations thereof generally refers to the ability of the film dosage form to rapidly imbibe moisture upon oral administration to a subject and immediately soften, whereby the subject is prevented from experiencing a prolonged adverse feeling in the mouth, and with respect to certain aspects of the disclosure refers to embodiments in which moisture (i.e., water) applied to a surface of the film penetrates the thickness of the film (e.g., typically about 5 μm to 200 μm) within 5, 10, 15 or 20 seconds. The wettability also ensures quick mucoadhesion ensuring the film sticks to the mucosa and stays in place.

The term “stable” refers to a product which exhibits no changes in the dissolution profile or remains within the established specifications and recovery when the product is exposed to long term or accelerated stability conditions (e.g., 25° C./60% RH and 40° C./75% RH) for an extended period of time while also demonstrating no or limited chemical degradation. The term “stable” can also refer also to mechanical stability, such as in the case where the product is recrystallizing; there will be a change in it flexibility and other mechanical properties. Additionally, term “stable” can also refer to the appearance of the film like color, color uniformity and texture.

Preferred film dosage forms include sublingual and buccal film oral dosage forms. Buccal and/or sublingual mucosa absorption allows the drug to be absorbed directly into the blood stream, skipping or significantly limiting the hepatic metabolism. From a pharmaceutical formulation perspective this is particularly challenging, as the process of transmucosal permeation needs to be carefully optimized to obtain an acceptable pharmacokinetic profile.

The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication or simply against constant washing of the saliva. This protective layer also serves to favor the directed absorption of the API within the oral cavity rather than enteric uptake in the gastrointestinal (GI) tract.

As used herein, the term “animal” is meant to indicate mammals, and to exclude humans.

The present disclosure provides methods and products for locally administering one or more active agents via adhesion of a film to a mucosae membrane such as, for example, mucosae membrane included in a human or animal mammalian oral cavity. A dissolving film containing an active agent is placed upon a mucosae membrane, such as a membrane within the oral cavity. The hydrophilic nature of the dissolving film causes the film to stick to the mucous membrane.

Δ9-Tetrahydrocannabinol (THC) is the primary psychoactive constituent of cannabis. Although predominantly smoked, oral administration occurs during illicit use and licit pharmacotherapy. Oral synthetic THC (dronabinol) is FDA-approved for treating nausea, vomiting, and anorexia, and there is strong interest in treatment with whole plant extracts containing THC and cannabidiol. Dronabinol also suppresses cannabis withdrawal and withdrawal-associated drug relapse.

Oral THC bioavailability is only 6%-10% available, and is influenced by vehicle due to gastric degradation and extensive first-pass metabolism. THC is rapidly oxidized to its active metabolite 11-hydroxy-THC (11-OH-THC) and further to THC COOH. Peak THC concentrations are lower after oral than smoked administration, but conversely, 11-OH-THC/THC ratios are higher after oral than smoked drugs.

The stability of different cannabinoids depends on the storage form and storage conditions. In the living plant, the precursors of THC and CBD are found in their acidic forms, THCA and CBDA. Neutral (THC, CBD) and acidic (THCA, CBDA) forms have different stability towards temperature and light exposure. Neutral cannabinoids are stable in the darkness at room temperature up to two weeks. However, exposure to light can lead to a significant decrease in the THC and CBD content. Acidic forms of cannabinoids decarboxylate and turn into neutral forms in both daylight and darkness when exposed to heat.

In the presence of atmospheric oxygen, THC is oxidized to CBN. Cannabidiol also undergoes chemical transformations when the pH is changed. Under acidic conditions it may transform into Δ9-THC by acid-catalyzed cyclization (8) and, under basic conditions in the presence of oxygen, it is oxidized to monomeric and dimeric hydroxyquinones. As such, the stability of the cannabinoid in the oral film is critical and complicated due to different temperatures and oxygen conditions the film is exposed to during manufacturing and storage.

For film formulation using cannabis derivatives such as THC and CBD, there are three main problems that arise: solubility/permeability, stability and mucoadhesion.

Relating to solubility and permeability, poor aqueous solubility is an important underlying factor of bioavailability since a drug cannot be absorbed through the buccal mucosa unless it is in a solution state or micronized but this often results in limited absorption. Many types of drugs show erratic absorption behavior with limited oral bioavailability. However, even if a given drug is solubilized it does not mean that it is absorbed, and accordingly permeation enhancers are often required.

Relating to stability, THC is metastable at room temperature, when exposed to air or light and its primary degradant is CBN. The oral film format promotes a larger surface area than other delivery options, meaning a bigger exposure of the drug.

Relating to mucoadhesion, it is defined as adhesion of material to mucus and/or an epithelial surface. Mucoadhesion occurs in two stages depending on the nature of dosage form and its delivery: Stage I (Contact Stage): wetting, spreading and swelling of the film surface creates close contact between a film and a membrane. Stage II (Consolidation Stage): moisture breaks molecules and interpenetration or dominant attractive interaction between two surfaces starts due to Vander walls forces, electrostatic attractions, hydrogen bonding and hydrophobic interactions. For complete bio adhesion attractive forces must overcome repulsive forces. The problem with cannabis derivatives is that the presence of non-polymeric excipients and fatty ingredients reduces the adhesion capability of the film and creates an oily surface, which prevents or impedes mucoadhesion.

The solution disclosed herein involves an equilibrium between solubilizing the cannabis using solvent(s) or an emulsion embedded in a matrix formulation that stabilizes but does not entrap the cannabis in mucoadhesive and stable film of acceptable weight.

Solubilizing the cannabis means that one or more solvent(s) capable of dissolving the cannabis during the blending step are used, and that the cannabis remains solubilized/amorphous once the solvent is removed during drying. For long-term stability, the cannabis must remain in the same solubilized form (amorphous) during the entire expected shelf life.

As disclosed herein, the use of emulsion systems are employed. Microemulsions are clear, thermodynamically stable, isotropic liquid mixtures of oil, water, and surfactant, usually in combination with a cosurfactant. The droplet size of the dispersed phase in a microemulsion is less than 100 nm. Nanoemulsions are typically prepared using two immiscible phases, and commonly exhibit a diameter of up to 500 nm. Spontaneous emulsification can also be used.

In such emulsion systems, the oil phase is dissolved in water-miscible or partially water-miscible organic solvents, such as acetone, ethanol or MEK.

The organic phase is poured into an aqueous phase containing surfactant to form spontaneous emulsion by rapid diffusion of organic solvent from the internal to the external phase, followed by direct solvent evaporation. The organic phase consists of Cannabis, Carrier oil, water-miscible organic solvent and lipophilic surfactant. The aqueous phase consists of water and hydrophilic surfactant.

Emulsion size is influenced by the surfactant concentration, oil phase composition, addition of co-surfactant and non-aqueous solvent and temperature, and will have an impact on absorption.

Self-nanoemulsifying drug delivery systems (SNEDDS) are anhydrous homogenous liquid mixtures. SNEDDS are a solubilization vehicles compromising oils, drug and emulsifiers, which form oil-in-water nano-scaled emulsion of approximately 200 nm or less in size upon dilution with water under gentle stirring. VESIsorb® (patented self-assembling colloidal droplet delivery system formulated to increase absorption), a lipid-based formulation that naturally self-assembles on contact with an aqueous phase into a colloidal system, it is a “nano”-scale solubilized system.

Upon adding VESIsorb® to an aqueous environment, it naturally self-assembles into a “colloidal emulsion,” where no energy is required to build up the emulsion droplets. Emulsifiers, oils and actives could move by “flip-flop” mechanism from one droplet to another or to other membranes (e.g. enterocytes).

The oral film containing the solubilized cannabis must exhibit mucoadhesion to ensure the film remains in close contact with the mucosa, in order to promote transmucosal absorption. Being a low permeable molecule, providing a longer time of contact between the mucosa and the film will promote a higher extend of absorption. A strong mucoadhesion is characterized by a resistance to lift the tongue when the film is position under the tongue or by the film staying in vertical position and not sliding down when the film is position on the inside of the cheek.

The formulations of this disclosure may be further applied for other cannabinoid compounds, including essential oils, terpenes, etc. The formulations of this disclosure may be further applied for other poorly soluble and poorly permeable drugs.

In other of its aspects, this disclosure provides formulations that may be adjusted and adapted for other routes of administration, including oral administration with gastric absorption.

The aim at hand is to develop and formulate an oral films of cannabis which is limited by its dissolution rate (BCS class II), for the direct absorption of drug via transmucosal lining to the systemic circulation. The proposed formulations in this disclosure have the potential to speed up dissolution, increase absorption and provide faster drug absorption from the oral mucosa area, which will provide quick onset of action. Two approaches were used to increase drug solubility and accelerate dissolution of cannabis oral films.

The first approach consists of preparing film using the solvent casting method with polymer soluble in alcohol, as film forming material and ethyl alcohol as casting solvent. Alcohol performs dual duties, it acts as casting solvent and in the same time, it converts the cannabis from a crystalline form to an amorphous form. With this approach, there are no need to improve the solubility of the cannabis before its incorporation into the film form.

The second approach consists of preparing fine colloidal dispersions of the cannabis such as emulsions or as a self emulsifying drug delivery system (SEDDS) and incorporating them into the polymer matrix to create the film.

Multiple ratios of oil, surfactant, and co-surfactant were explored to generate fine emulsion formulations. The appropriate ratio of drug, oil, surfactant and co-surfactant was determined by the inducing ability of the mixture to produce a fine emulsion. The dispersion of the different combinations in water was visually evaluated and the formulations presenting excellent emulsification capacity, giving a fine, stable at room temperature for extended period of time (evaluated on about 1 week) and translucide emulsion were selected.

To improve the matrix performance in term of physical properties, adhesion, onset of action and dissolution, different film matrices composed of different polymers and combination of polymers: PVP, HPC, HPMC, PEO and NaCMC, were prepared and evaluated on their mucoadhesion, dissolution rate and the onset of action.

The PVP-alcoholic formulation in example 1 showed that the onset of action occurred within the first 10 minutes. On the other hand, HPC/copovidone/HPMC based formulation, and the HPMC based formulation, in presence of MCT (example 5 and example 7, respectively) showed an onset of action between 10 and 15 minutes. As presented in Table 1, the rest of the formulations showed slower onsets.

TABLE 1

| Formulation | Onset of action | |
|---|---|---|
| F1 | <10 | min |
| F5 | 10 to 12 | min |
| F7 | 10 to 15 | min |
| F2 | 18 to 20 | min |
| F3 | 17 to 20 | min |
| F4 | <25 | min |
| F12 | 25 | min |
| F10 | 60 | min |
| F11 | 60 | min |
| F9 | >60 | min |
| F6 | >60 | min |
| F8 | >60 | min |

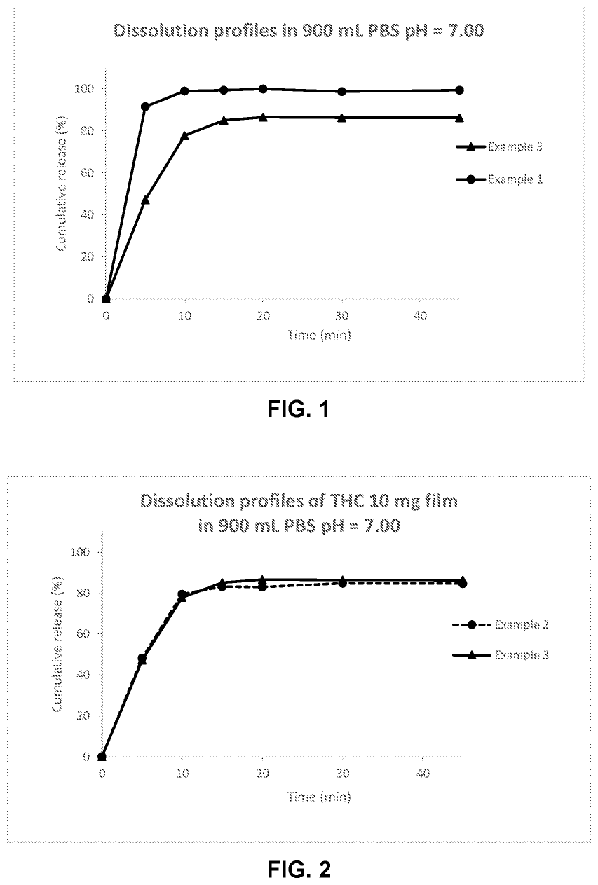

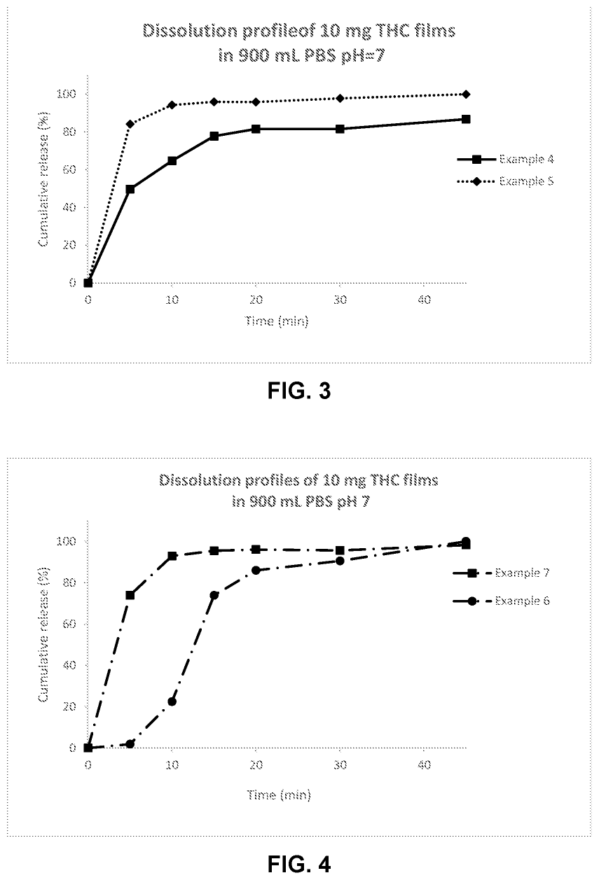

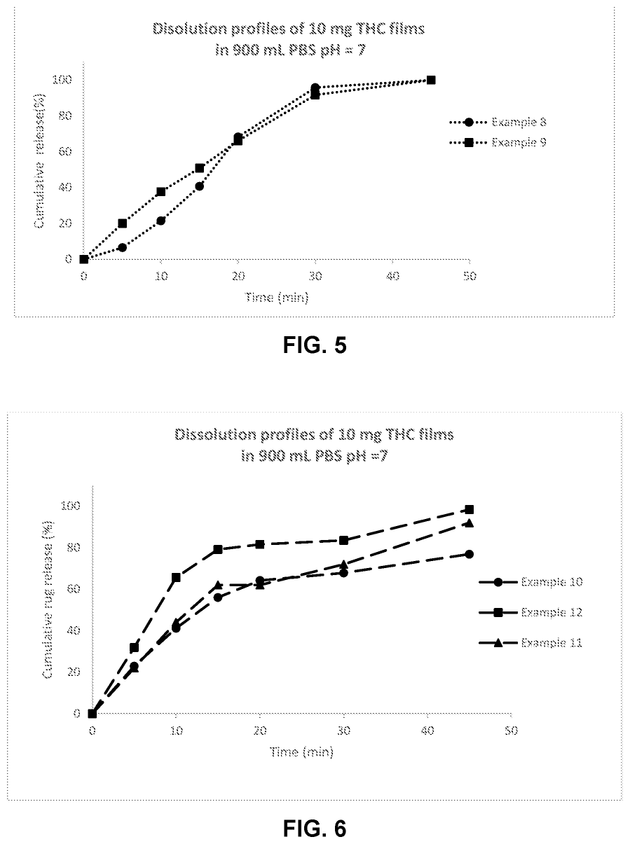

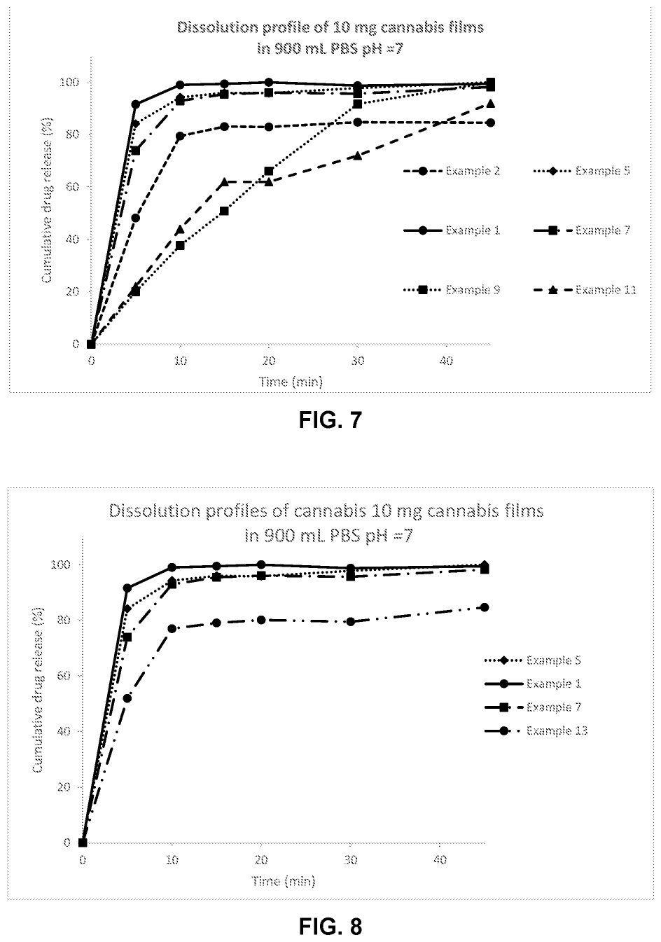

The dissolution profiles of the different formulations were tested and illustrated from FIG. 1 to FIG. 8. Notably, the dissolution experiments were performed in sink conditions, at RT and 50 rpm using 900 ml 50 mM phosphate buffer (pH 7) as the dissolution medium which is free of any surfactant. This eliminates any risk of influence of a surfactant not part of the formulation and gives to the method the ability to discriminate between the different formulations/batches that potentially have different quality attributes and in-vivo behavior.

During the development, it was observed that the dissolution rate of some formulations was affected by the presence of MCT and by the type of the solvent used in the blend and the hydrophilicity of the polymers used to generate the matrix. In particular, the selection of the casting solvent and the appropriate polymer type changed the formulation by promoting the solubility and the amorphous state, which increased the water affinity and solubility of cannabinoids.

The dissolution result, obtained for of the PVP-alcoholic based formulation in example 1 (99%) was significantly higher than PVP-emulsion based formulation in example 3 (79%) at 10 min, which correlates with the onset of action result. This clearly demonstrated that organic solvent promotes the solubility and the amorphous state for the cannabis, which increases the water affinity and solubility of the drug. In addition, the PVP in example 1, contributed to the stabilization of the amorphous state through specific interactions with the drug and elevating the glass transition temperature (Tg). It is also showed in dissolution profile comparison between example 2 and example 3 that the presence of MCT in PVP-emulsion-based formulation had no effect on dissolution improvement.

On the other side, example 5 and example 7, involving MCT with HPC/copovidone/HPMC (for example 5) and HPMC (for example 7) gave a significantly improved dissolution results (%) at 10 min (94% and 93%, respectively), in comparison with the formulations involving HPC/copovidone/HPMC without MCT (example 4) and HPMC without MCT (example 6), which provide dissolution rates at 10 minutes of 65% and 22%, respectively. This dissolution result is as high as that of the PVP-alcoholic formulation which also suggests an amorphous form of the drug. This result also correlates with the onset of action result which mentions an effect within the 15 first minutes. However, the presence of MCT in emulsion formulations with PVP (example 2), PEO (example 9) and CMC (example 12) does not seem appear to accelerate the dissolution, with an onset of action of 18, 60 and 25 minutes respectively.

The presence of PEO impacts negatively on the time to absorption by promoting entrapment and should be avoided, with an onset of action after one hour and a dissolution rate at 10 min of 37%.

It is suggested that the longer carbon side chain of HPC and HPMC interact with the emulsion of MCT-THC/water by forming hydrogen bond between the groups and at the emulsion surface. These groups combined with the back bone of the polymer can also form steric restriction effect between the emulsion droplets which reduces emulsion droplets aggregation. All these effects result in a more stable emulsion which disperse better in dissolution tests. HPMC and HPC/copovidone/HPMC also appears to act as precipitation inhibitors further improving the stability of the amorphous cannabis.

Based on the generated results a strong synergistic effect of dissolution enhancement involving MCT and the polymer at the same time is observed. It is well known that the mechanism of drug release from HPMC hydrophilic matrix is controlled by the hydration followed by swelling of the polymer to form a gel layer. HPMC tends first to swell and form a viscous gel which slowly dissolve promoting the drug diffusion from the matrix. This explains the low dissolution result in HPMC formulation in example 6 (in absence of MCT) at 10 minutes and the lag time of observed in the dissolution profile. However, the dramatic increase of the dissolution result of 99% at 10 minutes in presence of MCT, was unexpected since this synergistic effect between HPMC and MCT has not been revealed before.

On the other hand, it was also unexpected that the formulation involving Vesisorb® in example 13 was less effective than the formulations in example 1, example 5 and particularly example 7, using a composition of THC, MCT, Tween 80 and Span 80 at a weight ratio of 48.25/23/23/5.75%. Since it is known from literature that Vesisorb® forms cannabis microemulsions in biological media, with droplet sizes less than 100 nm, which would greatly increase the solubility and bioavailability of cannabis. This strongly suggested that the emulsifying system prepared in this disclosure will form a microemulsion once in the biological medium. It is also possible that this microemulsion forms in the oral cavity upon contact with saliva, which explains the facilitation of the absorption through the mucous membrane.

On the other hand, based on generated dissolution profile an unexpected correlation between in vitro dissolution efficiency and in vivo onset of action in most formulations was observed. In fact, it is known in the literature that emulsions have a poor in vitro/in vivo correlation due to a lack of predictive in vitro tests, issues in handling of liquid formulation, and physico-chemical instability of drug and/or vehicle components, which makes this in vitro dissolution method a relevant predictor of the in vivo performance of the drugs in development.

Finally, it was revealed that the formulation is physically and chemically very stable after 4 months of storage at room temperature. It was even surprising to note that formulations 14, 15 and 16, not containing carrier oil, co-surfactant and stabilizer, demonstrated no loss of cannabis content or any physical modification despite the non-use of the co-surfactant, Span 80 surfactant, MCT and stabilizer.

It was also observed in NaCMC matrix formulations (examples 10, 11 and 12) that the absorption of the cannabis was delayed when the matrix contains at least 10% sodium carboxymethyl cellulose (NaCMC) polymers high viscosity. The use of CMC as a matrix former must be limited to low viscosity grades like cellulose gum, or sodium carboxymethylcellulose (CMC), that has a viscosity of 25-50, a concentration of 2, and a spindle number of 1. The concentration must be maintained below 35% not to promote entrapment of the cannabis in the matrix which would results in a delayed action. The presence HPMC, NaCMC and the sodium alginate in the formulations exhibited a rapid and high mucoadhesion. The mucoadhesion can be assess by the resistance of the tongue to lift from the bottom of the mouth when the film is administered sublingually. This characteristic certainly contributes to the improvement of absorption during the first minutes due to the fact that the film is in intimate contact with the mucosa, which facilitates the diffusion of the drug into the mucosa. In addition, the presence of a highly soluble sugar like Maltitol with all the polymers appears to work synergistically and improve the mucoadhesion. This effect is likely attributable to quick dissolution and the creation of pores enabling a faster swelling of the polymer. However, when HPMC, sodium alginate and NaCMC were not employed, but the cannabis was in an amorphous state, the absorption was faster, however poor mucoadhesion was observed. Poor mucoadhesion is characterized by the film moving under the tongue and providing no resistance for the tongue. The lack of mucoadhesion appears to be linked to the cannabis presence itself and is exacerbated when lipophilic molecules are used to increase the permeability of the cannabis. The presence of solubilizer/permeation enhancer improves the cannabis absorption but also promotes some oiliness on the top of the film.

Hence, an equilibrium between the presence of amorphous cannabis, oil and mucoadhesive agents (Sodium alginate, HPMC or NaCMC) to reduce oiliness and promote adhesion, and a sugar to generate pores and ensure quick adhesiveness to the mucosa is required to obtain a quick onset of THC.

The fastest effect time is achieved with low viscosity NaCMC but must be in combination with a more viscous polymer to ensure some good mechanical properties and residence time to promote potential absorption. HPMC and a combination of PVP and HPMC/HPC also prevents entrapment while maintaining the THC amorphous. A pore former is required to ensure quick hydration of the matrix to promote adhesion.

Good Transmucosal Absorption

| | | |
|---|---|---|
| Amorphous | Low viscosity film | Presence of pore |
| cannabis | former with | former and |
| (emulsion or | emulsification | mucoadhesive |
| solvent) | properties | polymers |

Exemplary film formulations described herein are provided in examples 1 through 16. The following examples are provided to further clarify the present disclosure and are not to be interpreted as limitations of the present disclosure, as several variations of the present disclosure are possible without deviation from its spirit or scope.

Some ingredients of the examples are multifunctional ingredients. For example, PEG 400 can serve as solubilizer, co-solubilizer, permeation enhancer, co-surfactant and plasticizer.

| Function | Ingredients |
|---|---|
| Solubilizers/co-solubilzers | Ethanol, Medium chain triglycerides, glyceryl monolinoleate, Medium Chain |
| | Mono- and Diglycerides peppermint oil, vitamin E TPGS |
| | PEG 400, Propylen Glycol |
| Surfactants/co-surfactants | Tween 80, Tween 20, Span 80, Span 60, Propylen Glycol, PEG 400, Ethanol. |
| Permeation enhancers | PEG 400, Tween 80, Tween 20, vitamin E-TPGS, Ethanol |
| Sweeteners | Sucralose, maltitol, mannitol, sorbitol |
| Pore former, disintegrating agent | Maltitol, sodium alginate |
| Flavor | Menthol, peppermint oil |
| Plasticizer | PEG 200, PEG 300, PEG 400, PEG 600, Propylene Glycol, Glycerol |
| Anti-oxidant | Vitamin E-TPGS, ascorbic acid, EDTA, BHT |
| Preservative | Propyl paraben, methyl paraben, sodium benzoate, benzalkonium chloride, |
| | cetylpyridinium, ethanol, benzyl alcohol |
| Mucoadhesive agent | HPMC, sodium alginate, polycarbophil, xanthan, guar gum, carrageenan gum |
| Emulsifiers/emulsion stabilizer | Lecithin, xanthan, guar gum, carrageenan gum, sodium alginate |
| Viscosity agent | HPMC, Xanthan, quar gum, sodium alginate |
| Anti-foaming agent | Simethicone, ethanol |
| Film formers | NaCMC, PVP, copovidone, HPC, HPMC, PEO. |

In one embodiment, an oral dosage form comprising amorphous cannabis and a rapid release carrier system, wherein said dosage form is characterized by a discriminatory and biorelevant dissolution method performed in sink conditions, at room temperature, using paddle rotation of 50 rpm in 900 ml of a media composed of 50 mM phosphate buffer adjusted at pH 7 which is free of surfactant and providing: (i) A rapid release, wherein over 80% of the cannabis is released in less than 10 min. (ii) A high mucoadhesive property characterized by a resistance to lift the tongue when the film is administered sublingually or the vertical stability when administered on the inside of the cheek leading to high and rapid absorption and fast onset.

In one of its aspects, this disclosure provides a cannabinoid-loaded oral film formulations containing 1 to 25 w/t % of a cannabinoid but preferable less than 15% w/t %.

In some embodiments, the active ingredient comprises one or more cannabinoids.

In some embodiments, the active agent comprises at least one cannabinoid with at least one other non-cannabinoid active agent.

In some embodiments, the active ingredient comprises THC.

In some embodiments, the active ingredient comprises CBD.

In some embodiments, the active ingredient comprises CBD/CBDA.

In some embodiments, the active ingredient comprises THC/CBD

In some embodiments, the active ingredient comprises THC/THCA

In some embodiments, the active ingredient comprises CBC, CBG, CBDA, CBGA, CBDV, THCV, CBCV, CBGV or CBL.

In some embodiments, the cannabis is at least 75% amorphous.

In other aspects of this disclosure, the cannabis is at least 90% amorphous.

In one of its aspects, this disclosure provides an amorphous cannabinoid-loaded oral film formulations containing, at least one film former, at least one mucoadhesive polymer, at least one cannabis solubilzer, at least one hydrophilic surfactant, at least one co-surfactant, at least one permeation enhancer, at least one stabilizer, at least one flavor agent, at least one sweetening agent, at least one disintegrating agent and as a rapid release, wherein over 80% of the cannabis is released in less than 10 minutes.

In one embodiment, the oral dosage form has a solubilizer selected from the group of ethanol, Medium chain triglycerides, glyceryl monolinoleate or Medium Chain Mono- and Diglycerides.

In one embodiment, the oral dosage form has the film former polymer selected from the group of PVP, HPC, copovidone, HPMC, NaCMC, PEO and combinations thereof.

In some aspects of this disclosure, the mucoadhesive polymer is selected from the group of sodium carboxymethyl cellulos, polycarbophil, Sodium Alginate, HPMC, PEO and combinations thereof.

In one embodiment, the disintegrating agent is maltitol, sorbitol, lactose, sodium alginate and combinations thereof.

In one embodiment, the oral dosage form has a sweetener selected from the group of sucralose, mannitol, sorbitol, and combinations thereof.

In one embodiment, the oral dosage form has a co-solubilizer

In one embodiment, the oral dosage form has a co-solubilizer selected from the group of ethanol Propylene Glycol, PEG 300, PEG 400, PEG 600, Glycerol and combinations thereof.

In one embodiment, the oral dosage form further comprises a surfactant.

In one embodiment, the oral dosage form has a surfactant of Tween 20 or Tween 80 and/or combinations thereof.

In one embodiment, the oral dosage form has a co-surfactant.

In one embodiment, the oral dosage form has a co-surfactant selected from the group of Span 80, Span 60, Propylene Glycol, PEG 300, PEG 400, PEG 600, Glycerol, ethanol and combinations thereof.

In one embodiment, the oral dosage form has a permeation enhancer.

In one embodiment, the oral dosage form has a permeation selected from the group of PEG 400, PEG 300, PEG 600, Tween 20, Tween 80, vitamin E TPGS and combinations thereof.

In one embodiment, the oral dosage form has a plasticizer, wherein the plasticizer may consist of PEG 400, PEG 300, PEG 600, Glycerol, Propylene Glycol, and combinations thereof.

In one embodiment, the ratio of cannabis to polymers such as PVP, HPC, copovidone, NaCMC, HPMC and PEO is between about 1:6 to about 1:1.

In one embodiment, the ratio of cannabis to hydrophilic surfactant is between about 1:1 to about 1:3.

In one embodiment, the ratio of cannabis to lipophylic surfactant is between about 10:2 to about 10:1.

In one embodiment, the ratio of cannabis to carrier solubilizer such as Medium chain triglycerides, glyceryl monolinoleate or Medium Chain Mono- and Diglycerides is about 1:1 to about 2:1.

In one embodiment, the ratio of cannabis to permeation enhancer such as PEG 400 is about 1:3 to about 1:1.

In one embodiment, the ratio of cannabis to mucoadhesive agent such as sodium alginate and HPMC is between about 2:1 to about 3:1.

In one embodiment, the ratio of cannabis to disintegrating agent such as maltitol and sodium alginate is between about 4:1 to about 2:1.

In one embodiment, the oral dosage form further comprises a flavor.

In one embodiment, the flavor is selected from the group consisting of menthol, peppermint oil, limonene, and combinations thereof.

In one embodiment, the oral dosage form further comprises an anti-foaming agent

In one embodiment, the oral dosage form has an anti-foaming agent which may be simethicone or ethanol.

In one embodiment, the oral dosage form further comprises a stabilizer.

In one embodiment, the oral dosage form has a stabilizer selected from the group of vitamin E-TPGS, ascorbic acid, BHT, EDTA, and combinations thereof.

In one embodiment, the oral dosage form further comprises a preservative.

In one embodiment, the oral dosage form has a preservative that may be selected from the group of propyl paraben, methyl paraben, ascorbic acid, sodium benzoate, benzalkonium chloride, cetylpyridinium, ethanol, benzyl alcohol, and combination thereof.

In one embodiment, the ratio of cannabis to disintegrating agent such as maltitol and sodium alginate is between about 4:1 to about 2:1.

One aspect of this disclosure is a method of preparing a solvent system comprised of an aliphatic alcohol; adding an amount of cannabinoid that can be solubilized in the solvent system; adding an amount of co-solvent to achieve the maximal amorphization of the drug; adding polymers that dissolve in aliphatic alcohol and form a blend; adding permeation enhancer; adding flavour, adding sweeteners, adding antioxidant, adding preservative, adding surfactant and co-surfactant; and removing the solvent system from the blend to produce the oral film dosage form.

A second method of producing the oral dosage form is found in this disclosure, compromising: preparing colloidal cannabinoid delivery systems that do not require any sophisticated instruments, including the self-emulsification methods, (using liquid carrier oil (solubilizer), surfactants, co-surfactants, solvent and co-solvent, adding distilled water); adding polymer formers; adding permeation enhancer; adding flavour, adding sweeteners, adding pore formers, adding stabilizer, adding preservative and removing the solvent system from the blend to produce the oral film dosage form.

In some aspects of the methods above, the aliphatic alcohol is ethanol.

In some aspects of the methods above the ethanol soluble polymers are PVP polymer or copolymer and HPC.

In some aspects of the methods above the amount of PVP polymer or copolymer is from 1.0 g to 5.0 g per 15 to 30 mL of the solvent system.

According to some aspects of the disclosure, the oral dosage form further comprises a flavoring system.

In certain embodiments, it is advantageous to have a single layer film strip. However, in certain other embodiments, a multi-layer film strip is advantageous.

In certain embodiments, it was demonstrated a noticeable synergistic effect between MCT and HPMC, or MCT and HPC/copovidone/HPMC.

In certain embodiments, the ratio between MCT and HPMC is 1:10

In certain embodiments, the ratio between MCT and HPC/copovidone/HPMC is 1:6/2/2.

According to certain embodiments, the at least one active cannabinoid compound is derived biosynthetically from Cannabis plant species, from hemp, from plant-based or animal cell microorganisms, or is obtained by chemical synthesis.

According to certain embodiments, the formulation is physically and chemically stable at room temperature after 4 months of formulation and did not demonstrate any loss of cannabis content nor any physical modification.

In some embodiments, the surface area of the film ranges from about 4 cm 2 to about 7.5 cm 2. In some embodiments, the thickness of the film ranges from about 0.125 mm to about 0.175 mm.

In some embodiments, the oral film has a pleasant flavor for enhanced patient compliance.

In some embodiments, the oral film has a taste masker to mask the taste of the cannibinoid for enhanced patient compliance

In some embodiments, the oral film formulation comprises THC for the treatment of pain and CBD for the treatment of anxiety.

### Formulations and Preparations

### Example 1

In order to homogeneously incorporate the hydrophobic active into the formulation, ethanol is used as cannabinoid solubilizer and blending solvent, Tween 80 and PEG 400 are introduced as surfactant and co-surfactant/permeation enhancer to enhance the dissolution property of the drug and promote its release from the matrix once in contact with biological medium. PVP and HPC are added to the mix as film formers. Sweetening agents, flavoring agents, preservative, antioxidants and preservatives are also added to the mix. After mixing the blend is cast over liner rolls then subject to drying to form a film sheet.

Example 1

| Ingredients | % wet (w/w) | % dry (w/w) |
|---|---|---|
| Ethanol | 65.876 | — |
| Tw 80 | 2.196 | 6.435 |
| Cannabis | 3.953 | 11.583 |
| PEG 400 | 2.196 | 6.435 |
| Vit E-TPGS | 0.044 | 0.129 |
| Propylparaben | 0.044 | 0.129 |
| Menthol | 0.659 | 1.931 |
| Sucralose | 0.878 | 2.574 |
| HPC LF | 4.392 | 12.870 |
| PVP K90 | 19.763 | 57.915 |

### Example 2

In order to homogeneously incorporate the hydrophobic active into the formulation, cannabinoid-emulsion is prepared by mixing the liquid carrier solvent, the surfactant, the co-surfactant and the co-solvent until homogenous and translucent solution is obtained.

The mixing is carried out for between about 3-30 minutes at temperature of between about 25-70° C., to allow the full dissolution of the API and reduce the viscosity of the mix.

As a result, the emulsion composed of Cannabinoid, MCT, Tween 80 and Span 80 at a weight ratio of 48.25/23/23/5.75% was chosen, generating the more translucent or transparent which means the smallest droplet size and therefore better solubilization capacity, due to the increased surface area in contact with water.

In the aqueous phase, the plasticizer, PVP and HPC are combined. Sweetening agents, flavoring agents, antioxidant and disintegrating agent are then added to the mix.

After mixing the emulsified cannabinoid and polymer/water mixture, the wet blend is cast over liner rolls then subject to drying to form a film sheet.

Example 2

| | % wet | % dry |
|---|---|---|
| Ingredients | (w/w) | (w/w) |
| Purified water USP | 69.388 | — |
| Tw 80 | 1.633 | 5.333 |
| Span 80 | 0.408 | 1.333 |
| THC | 3.429 | 11.200 |
| PEG 300 | 2.449 | 8.000 |
| MCT oil | 1.633 | 5.333 |
| Menthol | 0.694 | 2.267 |
| Sucralose | 0.531 | 1.733 |
| Maltitol | 0.735 | 2.400 |
| PVP K90 | 14.694 | 48.000 |
| HPC L | 3.265 | 10.667 |
| Sodium Alginate | 1.143 | 3.733 |

### Example 3

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, PVP and HPC are combined. Sweetening agents, flavoring agents, antioxidant and disintegrating agent are then added to the mix.

After mixing the dispersed cannabinoid and polymer/water mixture, the wet blend is cast over liner rolls then subject to drying to form a film sheet.

Example 3

| | % wet | % dry |
|---|---|---|
| Ingredients | (w/w) | (w/w) |
| Purified water USP | 70.539 | — |
| Tw 80 | 1.660 | 5.634 |
| Span 80 | 0.415 | 1.408 |
| THC | 3.485 | 11.831 |
| PEG 300 | 2.490 | 8.451 |
| Menthol | 0.705 | 2.394 |
| Sucralose | 0.539 | 1.831 |
| Maltitol | 0.747 | 2.535 |
| PVP K90 | 14.938 | 50.704 |
| HPC L | 3.320 | 11.268 |
| Sodium Alginate | 1.162 | 3.944 |

### Example 4

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, HPC, copovidone and HPMC are combined. Sweetening agents, flavoring agents, preservative, antioxidant and disintegrating agent are then added to the mix.

After mixing the dispersed cannabinoid and polymer/water mixture, the wet blend is cast over liner rolls then subject to drying to form a film sheet.

Example 4

| | % wet | % wet |
|---|---|---|
| Ingredients | (w/w) | (w/w) |
| Water | 71.369 | — |
| Tw 80 | 1.847 | 6.452 |
| Span 80 | 0.462 | 1.613 |
| THC | 3.879 | 13.548 |
| PEG 400 | 3.879 | 13.548 |
| Vit E-TPGS | 0.046 | 0.161 |
| Propylen paraben | 0.046 | 0.161 |
| Menthol | 0.714 | 2.493 |
| Sucralose | 0.546 | 1.906 |
| Maltitol | 0.756 | 2.639 |
| HPC L | 8.396 | 29.326 |
| Copovidone | 4.198 | 14.663 |
| HPMC E5 | 1.343 | 4.692 |
| HPMC E50 | 1.343 | 4.692 |
| Sodium Alginate | 1.175 | 4.106 |

### Example 5

In order to homogeneously incorporate the hydrophobic active into the formulation, cannabinoid-emulsion is prepared by mixing the liquid carrier solvent, the surfactant, the co-surfactant and the co-solvent until homogenous and translucent solution is obtained.

The mixing is carried out for between about 3-30 minutes at temperature of between about 25-70° C., to allow the full dissolution of the API and reduce the viscosity of the mix.

In the aqueous phase, the plasticizer, HPC, copovidone and HPMC are combined. Sweetening agents, flavoring agents, antioxidant and disintegrating agent are then added to the mix.

After mixing the emulsified cannabinoid and polymer/water mixture, the wet blend is cast over liner rolls then subject to drying to form a film sheet.

Example 5

| | % wet | % dry |
|---|---|---|
| Ingredients | (w/w) | (w/w) |
| Water | 70.833 | — |
| Tw 80 | 1.667 | 5.714 |
| Span 80 | 0.417 | 1.429 |
| THC | 3.500 | 12.000 |
| PEG 400 | 3.500 | 12.000 |
| MCT oil | 1.667 | 5.714 |
| Vit E-TPGS | 0.042 | 0.143 |
| Propyl paraben | 0.042 | 0.143 |
| Menthol | 0.708 | 2.429 |
| Sucralose | 0.542 | 1.857 |
| Maltitol | 0.750 | 2.571 |
| HPC L | 8.333 | 28.571 |
| Copovidone | 4.167 | 14.286 |
| HPMC high viscosity | 1.333 | 4.571 |
| HPMC low viscosity | 1.333 | 4.571 |
| Sodium Alginate | 1.167 | 4.000 |

### Example 6

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, HPMC E5 and HPMC E15 are combined. Sweetening agents, flavoring agents, antioxidant, anti-foaming agent and disintegrating agent are then added to the mix.

After mixing the dispersed cannabinoid and polymer/water mixture, the wet blend is cast over liner rolls then subject to drying to form a film sheet.

Example 6

| | % wet | % wet |
|---|---|---|
| Ingredients | (w/w) | (w/w) |
| Water | 70.715 | — |
| Glycerol | 1.498 | 5.114 |
| Tw 80 | 1.664 | 5.682 |
| Span 80 | 0.416 | 1.420 |
| THC | 3.494 | 11.932 |
| PEG 400 | 3.494 | 11.932 |
| Vit E-TPGS | 0.042 | 0.142 |
| Propyl paraben | 0.042 | 0.142 |
| Menthol | 0.707 | 2.415 |
| Sucralose | 0.541 | 1.847 |
| Maltitol | 0.749 | 2.557 |
| HPMC E5 | 11.647 | 39.773 |
| HPMC E15 | 3.744 | 12.784 |
| Sodium Alginate | 1.165 | 3.977 |
| Simethicone | 0.083 | 0.284 |

### Example 7

In order to homogeneously incorporate the hydrophobic active into the formulation, cannabinoid-emulsion is prepared by mixing the liquid carrier solvent, the surfactant, the co-surfactant and the co-solvent until homogenous and translucent solution is obtained.

The mixing is carried out for between about 3-30 minutes at temperature of between about 25-70° C., to allow the full dissolution of the API and reduce the viscosity of the mix.

In the aqueous phase, the plasticizer, HPMC E5 and HPMC E15 are combined. Sweetening agents, flavoring agents, antioxidant, antifoaming agent and disintegrating agent are then added to the mix.

After mixing the emulsified cannabinoid and polymer/water mixture, the wet blend is cast over liner rolls then subject to drying to form a film sheet.

Example 7

| | % wet | % wet |
|---|---|---|
| Ingredients | (w/w) | (w/w) |
| Water | 69.558 | — |
| Glycerol | 1.473 | 4.839 |
| Tw 80 | 1.637 | 5.376 |
| Span 80 | 0.409 | 1.344 |
| THC | 3.437 | 11.290 |
| PEG 400 | 3.437 | 11.290 |
| MCT | 1.637 | 5.376 |
| Vit E-TPGS | 0.041 | 0.134 |
| Propyl paraben | 0.041 | 0.134 |
| Menthol | 0.696 | 2.285 |
| Sucralose | 0.532 | 1.747 |
| Maltitol | 0.736 | 2.419 |
| HPMC E5 | 11.457 | 37.634 |
| HPMC E15 | 3.682 | 12.097 |
| Sodium Alginate | 1.146 | 3.763 |
| Simethicone | 0.082 | 0.269 |

### Example 8

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, PEO and HPMC E50 are combined. Sweetening agents, flavoring agents, antioxidant and disintegrating agent are then added to the mix.

After mixing the dispersed cannabinoid and polymer/water mixture, the wet blend is cast over liner rolls then subject to drying to form a film sheet.

Example 8

| | % wet | % dry |
|---|---|---|
| Ingredients | (w/w) | (w/w) |
| Water | 76.278 | — |
| Tween 80 | 2.034 | 8.574491 |
| Span 80 | 1.017 | 4.287245 |
| PEG 400 | 2.543 | 10.71811 |
| Glycerol | 0.890 | 3.75134 |
| THC isolate | 2.543 | 10.71811 |
| Vit E-TPGS | 0.025 | 0.107181 |
| Propyl paraben | 0.025 | 0.107181 |
| Menthol | 0.458 | 1.92926 |
| Sucralose | 0.331 | 1.393355 |
| Maltitol | 0.432 | 1.822079 |
| PEO | 10.170 | 42.87245 |
| HPMC E50 | 2.543 | 10.71811 |
| Sodium Alginate | 0.712 | 3.001072 |

### Example 9

In order to homogeneously incorporate the hydrophobic active into the formulation, cannabinoid-emulsion is prepared by mixing the liquid carrier solvent, the surfactant, the co-surfactant and the co-solvent until homogenous and translucent solution is obtained.

The mixing is carried out for between about 3-30 minutes at temperature of between about 25-70° C., to allow the full dissolution of the API and reduce the viscosity of the mix.

In the aqueous phase, the plasticizer, PEO and HPMC E50 are combined. Sweetening agents, flavoring agents, antioxidant and disintegrating agent are then added to the mix.

After mixing the emulsified cannabinoid and polymer/water mixture, the wet blend is cast over liner rolls then subject to drying to form a film sheet.

Example 9

| | % wet | % dry |
|---|---|---|
| Ingredients | (w/w) | (w/w) |
| Water | 75.273 | — |
| Tween 80 | 2.007 | 8.118 |
| Span 80 | 1.004 | 4.059 |
| PEG 400 | 2.509 | 10.147 |
| Glycerol | 0.878 | 3.551 |
| THC isolate | 2.509 | 10.147 |
| MCT oil | 1.317 | 5.327 |
| Alpha tocopherol | 0.025 | 0.101 |
| Vit E-TPGS | 0.025 | 0.101 |
| Menthol | 0.452 | 1.826 |
| Sucralose | 0.326 | 1.319 |
| Maltitol | 0.427 | 1.725 |
| PEO | 10.036 | 40.589 |
| HPMC E50 | 2.509 | 10.147 |
| Sodium Alginate | 0.703 | 2.841 |

### Example 10

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, high viscosity NaCMC, low viscosity NaCMC, xanthan and H PMC are combined. Sweetening agents, flavoring agents, antioxidant and disintegrating agent are then added to the mix.

After mixing the dispersed cannabinoid and polymer/water mixture, the wet blend is cast over liner rolls then subject to drying to form a film sheet.

Example 10xample 10

| | % wet | % dry |
|---|---|---|
| Ingredients | (w/w) | (w/w) |
| Ethanol | 7.731 | — |
| Water | 73.444 | |
| Glycerol | 1.469 | 7.803 |
| Tw 80 | 1.546 | 8.214 |
| Span 60 | 0.580 | 3.080 |
| HPMC E15 | 0.618 | 3.285 |
| Xanthan | 1.546 | 8.214 |
| THC | 3.131 | 16.632 |
| Ascorbic acid | 0.039 | 0.205 |
| Sucralose | 0.503 | 2.669 |
| Maltitol | 03.015 | 16.016 |
| Menthol | 0.773 | 4.107 |
| NaCMC-Cekol 150 | 2.203 | 11.704 |
| NaCMC-Cekol 30 | 3.402 | 18.070 |

### Example 11

In order to homogeneously incorporate the hydrophobic active into the formulation, cannabinoid-micelle is prepared by mixing the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, low viscosity NaCMC, xanthan and HPMC are combined. Sweetening agents, flavoring agents, antioxidant and disintegrating agent are then added to the mix. After mixing the dispersed cannabinoid and polymer/water mixture, the wet blend is cast over liner rolls then subject to drying to form a film sheet.

Example 11

| | % wet | % dry |
|---|---|---|
| Ingredients | (w/w) | (w/w) |
| Ethanol | 7.911 | — |
| Water | 75.158 | — |
| Glycerol | 1.424 | 8.411 |
| Tw 80 | 1.582 | 9.346 |
| Span 80 | 0.396 | 2.336 |
| HPMC E50 | 0.633 | 3.738 |
| Xanthan | 1.345 | 7.944 |
| THC | 2.373 | 14.019 |
| Propyl paraben | 0.040 | 0.234 |
| Sucralose | 0.514 | 3.037 |
| Maltitol | 0.593 | 3.505 |
| Vitamine E | 0.119 | 0.701 |
| Menthol | 1.187 | 7.009 |
| NaCMC Ashland -7LF | 6.725 | 39.720 |

### Example 12

In order to homogeneously incorporate the hydrophobic active into the formulation, cannabinoid-emulsion is prepared by mixing the liquid carrier solvent, the surfactant, the co-surfactant and the co-solvent until homogenous and translucent solution is obtained.

The mixing is carried out for between about 3-30 minutes at temperature of between about 25-70° C., to allow the full dissolution of the API and reduce the viscosity of the mix.

In the aqueous phase, the plasticizer, high viscosity NaCMC, low viscosity NaCMC, xanthan and H PMC are combined. Sweetening agents, flavoring agents, antioxidant and disintegrating agent are then added to the mix.

After mixing the emulsified cannabinoid and polymer/water mixture, the wet blend is cast over liner rolls then subject to drying to form a film sheet.

Example 12

| | % wet | % dry |
|---|---|---|
| Ingredients | (w/w) | (w/w) |
| Ethanol | 7.593 | — |
| Water | 72.134 | — |
| Glycerol | 1.139 | 5.618 |
| Tw 80 | 1.519 | 7.491 |
| Span 80 | 0.569 | 2.809 |
| MCT oil | 1.936 | 9.551 |
| HPMC E15 | 0.607 | 2.996 |
| Xanthan | 1.519 | 7.491 |
| THC | 3.227 | 15.918 |
| Ascorbic acid | 0.038 | 0.187 |
| Sucralose | 0.494 | 2.434 |
| Maltitol | 2.961 | 14.607 |
| Menthol | 0.759 | 3.745 |
| Cekol 150 | 2.164 | 10.674 |
| Cekol 30 | 3.341 | 16.479 |

### Example 13

In order to homogeneously incorporate the hydrophobic active into the formulations, the cannabinoid is mixed to a self-assembled system VESIsorb®. The mixing is carried out for between about 3-30 minutes at temperature of between about 25-70° C., the heating is applied to allow the full dissolution of the API and reduce the viscosity of the mix.

In the aqueous phase, the plasticizer HPMC E5 and HPMC E15 are combined. Sweetening agents, viscosity agents, anti-foaming agent, flavoring agents, mucoadhesive agents, antioxidants, preservative and disintegrating agent are then added to the mix.
After mixing the self-emulsified cannabinoid and polymer/water mixture, the wet blend is cast over liner rolls then subject to drying to form a film sheet.

Example 13

| | % wet | % dry |
|---|---|---|
| Ingredients | (w/w) | (w/w) |
| Ethanol | 1.474 | — |
| Water | 66.333 | — |
| Vesisorb ® | 5.006 | 15.549 |
| PEG 400 | 3.286 | 10.206 |
| Menthol | 0.265 | 0.824 |
| THC isolate | 3.339 | 10.371 |
| Vit E-TPGS | 0.044 | 0.137 |
| Propyl paraben | 0.044 | 0.137 |
| Sucralose | 0.531 | 1.648 |
| Maltitol | 1.106 | 3.434 |
| HPMC E5 | 14.151 | 43.956 |
| HPMC E15 | 3.980 | 12.363 |
| Semithicone | 0.133 | 0.412 |
| Sodium Alginate | 0.310 | 0.962 |

### Example 14

In order to homogeneously incorporate the hydrophobic active into the formulation, cannabinoid-emulsion is prepared by mixing the liquid carrier solvent, the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, HPC and HPMC are combined. Sweetening agents, flavoring agents, and disintegrating agent are then added to the mix.

After mixing the emulsified cannabinoid and polymer/water mixture, the wet blend is cast over liner rolls then subject to drying to form a film sheet.

Example 14

| | % wet | % dry |
|---|---|---|
| Ingredients | (w/w) | (w/w) |
| Purified water USP | 68.569 | — |
| Tw 80 | 2.226 | 7.083 |
| THC Distillate | 4.675 | 14.875 |
| PEG 300 | 4.675 | 14.875 |
| Peppermint | 0.935 | 2.975 |
| HPC L | 11.220 | 35.699 |
| HPMC E5 | 6.234 | 19.833 |
| Sucralose | 0.935 | 2.975 |
| Maltitol | 0.530 | 1.686 |

### Example 15

In order to homogeneously incorporate the hydrophobic active into the formulation, cannabinoid-emulsion is prepared by mixing the liquid carrier solvent, the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, HPC and HPMC are combined. Sweetening agents, flavoring agents, and disintegrating agent are then added to the mix.

After mixing the emulsified cannabinoid and polymer/water mixture, the wet blend is cast over liner rolls then subject to drying to form a film sheet.

Example 15

| | % wet | % dry |
|---|---|---|
| Ingredients | (w/w) | (w/w) |
| Purified water USP | 72.864 | — |
| Tw 80 | 1.877 | 6.916 |
| CBD Distillate | 3.793 | 13.979 |
| PEG 300 | 2.870 | 10.578 |
| Peppermint | 0.795 | 2.929 |
| HPC L | 8.942 | 32.954 |
| HPMC E5 | 4.968 | 18.308 |
| Sucralose | 0.755 | 2.783 |
| Maltitol | 2.981 | 10.985 |
| Semithicone | 0.155 | 0.570 |

### Example 16

In order to homogeneously incorporate the hydrophobic active into the formulation, cannabinoid-emulsion is prepared by mixing the liquid carrier solvent, the surfactant, the co-surfactant and the co-solvent until homogenous solution is obtained.

The mixing is carried out for between about 3-30 minutes at temperature of between about 25-70° C., to allow the full dissolution of the API and reduce the viscosity of the mix.

In the aqueous phase, the plasticizer, HPC and HPMC are combined. Sweetening agents, flavoring agents, and disintegrating agent are then added to the mix.

After mixing the emulsified cannabinoid and polymer/water mixture, the wet blend is cast over liner rolls then subject to drying to form a film sheet.

Example 16

| | % wet | % dry |
|---|---|---|
| Ingredients | (w/w) | (w/w) |
| Purified water USP | 68.337 | — |
| Tw 80 | 2.151 | 6.795 |
| CBD Distillate | 2.541 | 8.026 |
| THC Distillate | 2.364 | 7.466 |
| PEG 300 | 3.290 | 10.392 |
| Peppermint | 0.911 | 2.878 |
| HPC L | 10.251 | 32.374 |
| HPMC E5 | 5.695 | 17.986 |
| Sucralose | 0.866 | 2.734 |
| Maltitol | 3.417 | 10.791 |
| Semithicone | 0.177 | 0.560 |

The above description is considered that of the preferred embodiment(s) only. Modifications of these embodiments will occur by those skilled in the art and by those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1- An oral film comprising amorphous cannabis and a rapid release carrier system, wherein said film is characterized by a discriminatory and biorelevant dissolution method performed in sink conditions, at room temperature, using paddle rotation of 50 rpm in 900 ml of a media composed of 50 mM phosphate buffer adjusted at pH 7 which is free of surfactant and providing:
(i) a rapid release, wherein over 80% of the cannabis is released in less than 10 min.
(ii) a high mucoadhesive property characterized by a resistance to lift the tongue when the film is administered sublingually or the vertical stability when administered on the inside of the cheek leading to high and rapid absorption and fast onset.

2- The oral film of claim 1, containing 1 to 25 with % of cannabis but preferably less than 15% with %.

3- The oral film of claim 1, wherein the cannabis comprises one or more cannabinoids, including but not limited to THC, CBD, CBDA, CBN, CBG, or a combination thereof.

4- The oral film of claim 1, wherein the cannabis is at least 75% amorphous.

5- An amorphous cannabinoid-loaded oral film formulation comprising, at least one film former polymer, at least one mucoadhesive polymer, at least one solubilizer, at least one hydrophilic surfactant, at least one co-surfactant, at least one permeation enhancer, at least one stabilizer, at least one flavor agent, at least one sweetening agent, at least one disintegrating agent and as a rapid release, wherein over 80% of the cannabinoid is released in less than 10 min.

6- The oral film of claim 5, wherein the solubilizer is selected from the group of ethanol, medium chain triglycerides, glyceryl monolinoleate or medium chain Mono- and Di-glycerides.

7- The oral film of claim 5, wherein the film former polymer is selected from the group of PVP, HPC, copovidone, HPMC, NaCMC, PEO and combinations thereof.

8- The oral film of claim 5, wherein the mucoadhesive polymer is selected from the group of sodium carboxymethyl cellulose, polycarbophil, Sodium Alginate, HPMC, PEO and combinations thereof.

9- The oral film of claim 5, further comprising a co-solubilizer, wherein the co-solubilizer is selected from the group of ethanol, Propylene Glycol, PEG 300, PEG 400, PEG 600, Glycerol and combinations thereof.

10- The oral film of claim 5, further comprising a surfactant optionally in combination with a co-surfactant.

11- The oral film of claim 5, further comprising a permeation enhancer, wherein the permeation enhancer is selected from the group of PEG 400, PEG 300, PEG 600, Tween 20, Tween 80, vitamin E TPGS and combinations thereof.

12- The oral film of claim 5, further comprising a plasticizer, wherein the plasticizer is selected from the group of PEG 400, PEG 300, PEG 600, Glycerol, Propylene Glycol, and combinations thereof.

13- The oral film of claim 5, wherein the ratio of cannabinoid to polymers such as PVP, HPC, copovidone, NaCMC, HPMC and PEO is between about 1:6 to about 1:1.

14- The oral film of claim 5, wherein the ratio of cannabinoid to hydrophilic surfactant is between about 1:1 to about 1:3.

15- The oral film of claim 5, wherein the ratio of cannabinoid to lipophylic surfactant is between about 10:2 to about 10:1.

16- The oral film of claim 5, wherein the ratio of cannabinoid to carrier solubilizer such as Medium chain triglycerides, glyceryl monolinoleate or Medium Chain Mono- and Diglycerides is about 1:1 to about 2:1.

17- The oral film of claim 5, wherein the ratio of cannabinoid to permeation enhancer such as PEG 400 is about 1:3 to about 1:1.

18- The oral film of claim 5, wherein the ratio of cannabinoid to mucoadhesive agent such as sodium alginate and HPMC is between about 2:1 to about 3:1.

19- The oral film of claim 5, further comprising a stabilizer, wherein the stabilizer is selected from the group of vitamin E-TPGS, ascorbic acid, BHT, EDTA, and combinations thereof.

20- The oral film of claim 5, further comprising a preservative, wherein the preservative is selected from the group of propyl paraben, methyl paraben, ascorbic acid, sodium benzoate, benzalkonium chloride, cetylpyridinium, ethanol, benzyl alcohol, and combinations thereof.

21- The oral film of claim 5, wherein the ratio of cannabinoid to disintegrating agent such as maltitol and sodium alginate is between about 4:1 to about 2:1.

22- A method of producing the oral film of claim 1, comprising: preparing a solvent system comprised of an aliphatic alcohol; adding an amount of cannabinoid that can be solubilized in the solvent system; adding an amount of co-solvent to achieve the maximal amorphization of the drug; adding polymers that dissolve in aliphatic alcohol and form a blend; adding permeation enhancer; adding flavour, adding sweeteners, adding antioxidant, adding preservative, adding surfactant and co-surfactant; and removing the solvent system from the blend to produce the oral film dosage form.

23- The method of claim 22, wherein the aliphatic alcohol is ethanol, wherein the ethanol soluble polymers are PVP polymer or copolymer and HPC, and wherein the amount of PVP polymer or copolymer and HPC is from 1.0 g to 5.0 g per 15 to 30 mL of the solvent system.
